(19) United States
(12) Patent Application Publication
(10) Pub. No.: US 2013/0123227 A1
(43) Pub. Date: May 16, 2013

(54) NOVEL ORGANO-PALLADIUM COMPLEXES

(71) Applicant: Merrill Garnett, East Islip, NY (US)

(72) Inventor: Merrill Garnett, East Islip, NY (US)

(21) Appl. No.: 13/639,698

(22) PCT Filed: Apr. 12, 2011
(86) PCT No.: PCT/US11/32114

§ 371 Date: Jan. 23, 2013

Related U.S. Application Data
(60) Provisional application No. 61/323,543, filed on Apr. 13, 2010.

(51) Int. Cl. A61K 31/555; C07F 15/00

(52) U.S. Cl. CPC A61K 31/555; C07F 15/006
USPC 514/184; 549/3

(57) ABSTRACT

The present invention relates generally to novel complexes of palladium or a palladium salt, lipoic acid and a long chain fatty acid, where the palladium is bonded to lipoic acid via both sulfurs and carboxyl group oxygens of lipoic acid, and where the long chain fatty acid is bonded to the palladium via the carboxyl group of the long chain fatty acid. The complexes are useful as cancer chemotherapy agents.

### CROSS-REFERENCE TO RELATED APPLICATION

This application claims the benefit of U.S. Provisional Application No. 61/323,543, filed Apr. 13, 2010, which is incorporated herein by reference in its entirety.

### BACKGROUND OF THE INVENTION

The present invention relates generally to novel organo-palladium complexes and pharmaceutical compositions comprising the same. The novel complexes are useful as cancer chemotherapy agents.

A number of researchers have studied the role that nucleic acids play in cancer. Although electron reduction of single nucleotides has been reported, previous reviews of nucleic acids and their role in cancer make little mention of reactions or substances which result in the electron reduction of DNA or RNA.

The first redox regulation of the transcription of the proto-oncogenes c-fos and c-jun was reported in 1990. It was hypothesized that nuclear factor reduces a critical cysteine residue in fos and jun that is required for DNA-binding activity. It was believed that one or more cysteine residues in fos and jun are important for DNA binding and that reduction is required for association with DNA.

The findings thus suggested that modification of the redox state of fos and jun may contribute to the formation of specific protein-DNA complexes. The bacterial transcriptional regulatory protein, Oxy R, which regulates gene expression in response to oxidative stress, was found to change DNA-binding specificity depending on the redox state. Thus, regulation by reduction-oxidation was believed to be a mechanism of control for certain transcription factors.

Subsequently, the free energy of formation of relaxed trefoil and figure-eight DNA knots was studied. Supercoiled trefoil DNA knots were also evaluated. It was found that the presence of a knot in a relaxed or supercoiled DNA ring is associated with a substantial free energy cost. Furthermore, in enzyme-catalyzed reactions that yield knotted products, this free energy cost must be compensated for by a favorable free energy term, such as that derived from protein-DNA interactions.

Palladium complexes for cancer therapy have been reviewed. Although palladium has not been considered a physiologic substance, its contributions to hydrogen storage and electron transfer have been reported.

Certain palladium compounds have been described as inhibitors of growth, and have been shown to be interactive or able to bind with DNA. Such working concepts of growth inhibition are quite general and the mechanism of disease specificity has not been further approached.

Palladium lipoic acid (PdLA) complexes and methods for using them in the treatment of tumors and psoriasis have been disclosed in U.S. Pat. Nos. 5,463,093, 5,679,697 and 5,776,973.

Despite advancements in the art, there remains a need for novel cancer treatments and anti-cancer compounds.

### BRIEF DESCRIPTION OF THE FIGURES

FIG. 1a—MCF-7 breast cancer cells and their residual in vitro cell-mass absorbances as a response to G10Z (right side), and G10-AC (left side) by concentration (shorter bars with higher drug concentration show more cytotoxicity). The five test concentrations are graded from 3.0 μg/mL to 48.0 μg/mL.

FIG. 1b—H-80 brain cancer cells and their in vitro cell-mass absorbances as a response to G10Z (right side), and G10-AC (left side) by concentration (shorter bars with higher concentrations show more cytotoxicity). The five test concentrations are graded from 3.0 μg/mL to 48.0 μg/mL.

FIG. 2a—Cell mass absorbances show cytotoxic effect of G10Z (right side of graph), and G10Z with vitamin B12 (left side of graph), on MCF-7 breast cancer cells. Toxicity is dose dependent. Cytotoxicity is G10Z dose dependent with gradations from 1-17×10⁻⁵ mg/ml. Concentrations of B12 arc in 5 gradations from 1-17×10⁻⁴ μg/ml. Lowest G10Z dose shows a cooperative cytotoxic effect for B12 starts at 10⁻⁴μg/ml B12.

FIG. 2b—Cell mass absorbances show cytotoxic effect of G10Z (right side of graph), and G10Z with vitamin B₁₂ (left side of graph), on H-80 brain tumor cells. Toxicity is dose dependent. Cytotoxicity is G10Z dose dependent with gradations from 1-17×10⁻⁵ mg/ml. Concentrations of B12 arc in 5 gradations from 1-17×10⁻⁴ μg/ml. Lowest G10Z dose shows a cooperative cytotoxic effect for B12 starts at 10⁻⁴ μg/ml B₁₂.

FIG. 3a Ehrlich carcinoma monolayer culture.

FIG. 3b Ehrlich carcinoma culture with G10Z at 10⁻⁶ M shows heterochromatin and rounded up cells.

FIG. 4 Mott-Schottky analysis shows G10Z undergoes frequency dependent inductance oscillation (20 [blue], 40 [green], 60 [red], 100 [brown], and 200 [black] mhz.). Vertical axis is inductance, horizontal axis is voltage. Lower frequencies produce greater amplitudes of inductance oscillation.

FIG. 5—Mott-Schottky analysis shows inductance oscillation of G10Z alone (red), and then with added DNA (blue)—producing a shift in the voltage range. This suggests a resonant reaction occurs.

FIG. 6—Voltammetry shows G10Z (red), is oxidized by DNA (blue), inducing a new peak (black). This shift is 60. mv. towards the (+) pole.

FIG. 7—Electron spin resonance signal generated by DNA and G10Z+B12 in saline. The spin quartet is marked by the bracket. Hyperfine splitting distances arc 6.5 Gauss.

FIG. 8a Pseudo-chromosome pattern induced by G10Z on DNA/NaCl.

FIG. 8b Pseudo-chromosome pattern induced by G10Z on DNA/NaCl—showing fibrillar structure.

FIG. 9 a DNA/histone IIS/NaCl. Liquid crystal shows straight filaments with no twisted forms.

FIG. 9 b G10Z induces a twisted cable structure on DNA/histoneIIS/NaCl—suggesting G10Z is a rotational oscillation vector.

FIG. 10 Normal vine growth pattern of the sporulating mold Dictyostelium Discoideum.

FIGS. 11—G10Z induces an abundant orchard pattern of spore proliferation in Dictyostelium Discoideum.

FIG. 12—Bakers' Yeast-control

FIG. 13—Induction of heterochromatin (dense chromatin) by G10Z (10⁻⁶M) in Bakers' yeast.

FIG. 14—UV-Visible spectra of G10Z. Molar absorbance coefficients are 11,450 (237.5 nm), and 9,350 (284 nm).

FIG. 15a-FTIR spectra of Pd-lipoic acid, and arginine-linolcic acid, and their reaction product- G10. There is a loss of the arginine-linoleic minimum at 2350 cm⁻¹, and its conversion to a small peak (arrows).

FIG. 15b—FTIR of Zinc-threonine complex.

FIG. 15c—FTIR signature of G10Z. The CH2 bending vibration minimum from arginine-linoleic solution is shifted to 2359 cm⁻¹. Representative peaks (cm⁻¹) are 1052, 1153, 1314, 1411, 1567, 2857, and 2930. G10 peaks at 750 and 2350 cm⁻¹ are suppressed in G10Z.

FIG. 16—Liquid crystal DNA (ct) in NaCl shows a small native waveform

FIG. 17—Liquid crystal DNA in NaCl doped to 10⁻³M. G10Z, shows an increase in length of the waveforms. This resembles a Doppler effect.

FIG. 18—Liquid crystal of polydeoxy guanidylic cytidylic acid (poly GC) in NaCl shows no native waveforms.

FIG. 19—Liquid crystal of poly (GC) in NaCl doped to 10⁻³M. G10Z shows waveform induction.

FIG. 20—Liquid crystal form of zinc tri-threonine in NaCl shows uniform orientation of layered subunits. The pattern resembles a physical model of an array of closely packed plates as in an electronic device (18).

FIG. 21—Electrophoresis of G10Z (brown streak on left) shows more rapid downward migration, and therefore a lower molecular weight than the bradykinin marker (molecular weight 1060=blue spot on right). The G10Z molecular weight is calculated to be near 897: (1 palladium+1 lipoic acid+1 linoleic acid+1 zinc+2 threonines).

FIG. 22a—When palladium tetrachloride is combined with excess NaOH, and dried on a microscope slide, a periodic packing pattern shows. This is attributed to the coupling of palladium spin oscillations to the bound water. This is an example of a liquid crystal solvent lattice (phase microscopy LP).

FIG. 22b—G10Z shows liquid crystal stars in 0.1 M NaCl (phase microscopy HP).

FIG. 23—HPLC-Large peak of G10Z with a small peak on right from excess threonine ligand.

### SUMMARY OF THE INVENTION

The present invention provides a novel complex of palladium or a palladium salt, lipoic acid and a long chain fatty acid, wherein the palladium is bonded to lipoic acid via both sulfurs and carboxyl group oxygens of lipoic acid, and wherein the long claim fatty acid is bonded to the palladium via the carboxyl group of the long chain fatty acid.

In one embodiment of the invention, the palladium salt is selected from the group consisting of palladium chloride, palladium bromide, palladium iodide, palladium nitrate, palladium oxide and palladium sulfide.

In another embodiment of the invention, the lipoic acid comprises a lipoic acid derivative having a carbon chain of between 2 and 20 carbon atoms. A complex is also provided wherein the lipoic acid is a lipoic acid derivative having a side group or groups selected from the group consisting of carboxyl, sulfur and amine.

The present invention provides a complex wherein the lipoic acid derivative is lipoamide. Additionally, the invention provides a complex wherein the long chain fatty acid is linolcic acid or docosahcxaenoic acid.

In one embodiment of the invention, the complex further comprises zinc, and the zinc is bonded to the methyl of the long chain fatty acid.

In yet another embodiment of the invention, the complex further comprises one or two amino acids, wherein the amino acid(s) are bonded to the zinc. In addition, a complex having two amino acids bonded to zinc is provided. In a preferred embodiment, the amino acids are both threonine.

The present invention provides a pharmaceutical composition of matter comprising a pharmaceutically effective amount of a complex of palladium or a palladium salt, lipoic acid and a long chain fatty acid, wherein the palladium is bonded to lipoic acid via both sulfurs and carboxyl group oxygens of lipoic acid, and wherein the long claim fatty acid is bonded to the palladium via the carboxyl group of the long chain fatty acid, and a pharmaceutically acceptable carrier.

In one embodiment of the invention, the pharmaceutical composition contains the complex that further comprises zinc, and the zinc is bonded to the methyl of the long chain fatty acid.

In yet another embodiment of the invention, the pharmaceutical composition contains the complex that further comprises one or two amino acids, wherein the amino acid(s) are bonded to the zinc. In addition, a complex having two amino acids bonded to zinc is provided. In a preferred embodiment, the amino acids are both threonine.

In a final embodiment of the invention, the pharmaceutical composition contains the palladium-lipoic acid complex in the form of a solution, and in an amount sufficient to obtain a concentration of about 0.04 M.

### DETAILED DESCRIPTION OF THE INVENTION

The present invention provides novel palladium-lipoic acid complexes that are useful for the treatment of cancer, i.e. tumorigenesis. Surprisingly, it has been discovered that by altering the enzymatic or catalytic pathway represented by even a singular or very few gene sites, the native conformation of large tracts of DNA can be altered. The present invention is based on the surprising discovery that electron energy from a normal metabolic hydrogen carrier, such as lipoic acid, can be shunted to nucleic acids. The electron energy which is shunted can be measured by conventional voltammetric means.

The present invention is based on the discovery that specific forms of electronic energy transfer to DNA are mechanisms to condense tumor cell chromatin. Normal cells have condensed chromatin except in inflammatory conditions. Chromatin is poorly condensed in the malignant state, e.g. tumor cells. It is noted that there is a documented mitochondrial respiratory deficiency in tumors which is measured as a failure of oxygen uptake.

In consideration of this tumor respiratory deficiency, there are implications and reports of deficiency in the tumor mitochondrial electron oxygen chain. This implies a deficiency in tumor mitochondrial electron transfer.

Based on the above, the present invention relates to the introduction of novel organo-palladium complexes and their ability to induce electron transfer to tumor systems. The present invention also relates to the transfer of electron energy from a normal metabolic hydrogen carrier to nucleic acids. DNA has previously been described as an intermediate for electron transfer reactions. In particular, it has been reported that a double-stranded DNA polymer can mediate long-range electron transfer between bound donor-acceptor pairs.

The present inventor has found that a novel palladium-lipoic acid complex of the present invention can function as a polynucleotide reductase to transfer electrons into DNA and RNA. Further, and without wishing to be bound by any theory, the present inventor believes that when the electron energy from a palladium-lipoic acid complex of the present invention is shunted to DNA or RNA, it alters the nucleic acid configuration.

A polynucleotide reductase capable of shunting electron energy from itself to DNA is termed a DNA reductase, while a polynucleotide reductase capable of shunting electron energy from itself to RNA is termed a RNA reductase.

The complexes of the invention may be identified using UV-visible spectroscopy, and preferably by cyclic voltammetry, as discussed further in the Examples. The structures of these complexes, as shown in the Examples, were also studied by Fourier transform-infrared spectroscopy (FTIR). Cyclic voltammetry was performed to demonstrate the charge interactions of the complexes with DNA or RNA. These studies illustrated that the complexes of the present invention shunt electron energy from the complexes to nucleic acids of DNA or RNA and are polynucleotide reductases. The results of these studies are further discussed in the Examples.

The complex of the invention may exist in a solid form, however, the complex is preferably in a liquid form as a dispersion, or more preferably as a solution. The complex, also referred to as a coordination compound herein, is a compound containing a metal atom or ion bonded by at least one ionic bond to a number of anions or molecules. The complexes of the present invention comprising a transition metal ion are thermodynamically stable.

As is common in metal to ligand syntheses, multiple complexes of palladium with lipoic acid may be produced. The general formula of the complex of the present invention is (palladium)ₙ(lipoic acid)ₙ(long chain fatty acid)ₙ wherein n=1. In a preferred embodiment of the invention, the general formula of the complex is (palladium)ₙ(lipoic acid)ₙ(long chain fatty acid)ₙ(zinc)ₙ(amino acid)ₘ, wherein n=1 and m=1.

An exemplary structure of the complex is as follows (G10Z):

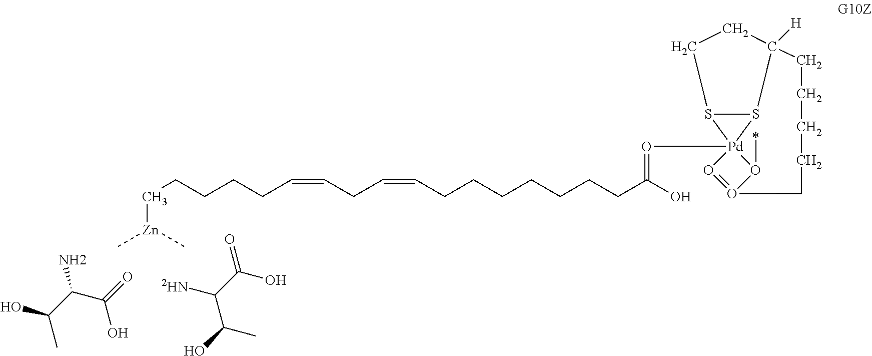

The bonds of the palladium-lipoic acid complex are coordinate covalent. More specifically, studies have shown that the palladium-lipoic complex is bonded by coordinate covalent bonds: (1) at the carbonyl end of the substituent having a carboxyl group with probable resonance involvement of both oxygens, and (2) at one or more sulfur atoms. The palladium adds a bond to the carboxyl end of the long chain fatty acid (e.g. linolcic acid, shown for example). The methyl end of the fatty acid is bonded to zinc. The zinc is also amide coordinated to one or two amino acids (e.g. threonines, shown for example).

The lipoic acid in the complex comprises a bent carbon chain with the ends of the chain bonded to the palladium. The above structure is represented above as a bent cyclic structure. However, while the figure shows a planar structure, crystallographic studies as discussed below show the structure to be three-dimensional with the palladium in the center of the complex.

### Lipoic Acid

As previously stated, lipoic acid is one component of the complex of the present invention. The present inventor has found that lipoic acid and its derivatives are highly specific for transferring electron energy from a normal metabolic hydrogen carrier to nucleic acids. Lipoic acid occurs in an oxidized or disulfide form, or in a reduced or dithiol form. The structure of lipoic acid in its oxidized form is as follows:

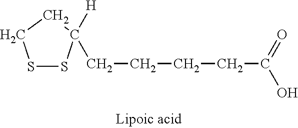 [SMILES: O=C(O)CCCCC1CCSS1]

The structure of the reduced form of lipoic acid, i.e., dihydrolipoic acid, is as follows:

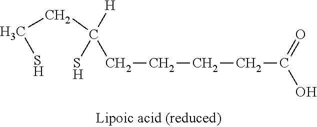 [SMILES: O=C(O)CCCCC(S)CCS]

As can be seen by the above structures, lipoic acid has a long, flexible side chain, which enables it to rotate from one active site to another in enzyme complexes. As shown in Campbell et al., Biochemistry Illustrated, 2d, Churchill Livingstone, 126 (1988), lipoic acid is a hydrogen carrier and an acetyl-group carrier for the decarboxylation of pyruvic acid. Lipoic acid is then present as acetyllipoic acid, having both an acetyl group and a hydrogen atom. In the pyruvic decarboxylation reaction, the acetyl group is donated to CoA and the H is donated to NAD+.

Derivatives of lipoic acid, in either its oxidized or reduced form, may also be used in the practice of the present invention, including lipoic acid analogues having a shortened or lengthened carbon chain, e.g., the lipoic acid derivative may comprise a carbon chain of at least 2 carbon atoms, preferably a C₂ to C₂₀ hydrocarbon chain, and most preferably a C₄ to C₁₀ hydrocarbon chain. Lipoic acid derivatives having one to three additional side groups, e.g., carboxyl, sulfur or amine groups may also be used. The side groups may be attached, for example, to one of the sulfur atoms, along the carbon chain, or may be substituted for the hydroxyl group at the carbonyl end of the lipoic acid moiety. A particularly preferred lipoic acid derivative is, for example, lipoamidc.

In addition, the palladium-lipoic acid complex of the present invention may further comprise at least one ligand to the palladium-lipoic acid complex. For example, the additional ligand to the palladium-lipoic acid complex may be an inorganic anionic ligand, including without limitation acetate, acetylacetonate, amine, ammonium chloride, ammonium nitrate, bromide, chloride, fluoride, iodide, nitrate, nitrite, oxalate, oxide, pyridine, sulfate and sulfide. The lipoic acid derivative may further comprise additional cations, for example, sodium, potassium, magnesium, calcium, ammonia, vanadate, molybdate, zinc and tin. Furthermore, the lipoic acid of the complex of the present invention may be present in its reduced or oxidized form.

Other derivatives of lipoic acid known in the art may also be used in the practice of the present invention. The derivatives are suitable if the ability to transfer electron energy from a normal hydrogen carrier to a nucleic acid is retained. As used in the present specification, the term “lipoic acid” is intended to include the derivatives specifically identified supra as well as other derivatives known in the art. The features of lipoic acid believed to be necessary for the present invention include at least two sulfur atoms, a hydrocarbon chain having a length of two to twenty carbon atoms, and one or more carboxyl groups.

### Palladium

The metal ion of the novel complex of the present invention is palladium. Palladium is a transition metal of Group VIII of the periodic table. Salts of palladium may also be employed in preparing the Pd-lipoic acid complexes of the present invention. The palladium salts may be selected from, and are not limited to, for example, palladium acetate, palladium acetylacetonate, palladium ammonium chloride, palladium ammonium nitrate, palladium bromide, palladium chloride, palladium diamine nitrite, palladium diamylaminc nitrite, palladium dibromide, palladium difluoride, palladium dioxide, palladium dipyridine nitrite, palladium ethylenediamine nitrite, palladium iodide, palladium monoxide, palladium nitrate, palladium oxalate, palladium oxide, palladium sulfate, palladium sulfide, palladium tetramine dichloride, palladous potassium bromide, palladous potassium chloride, palladous sodium bromide, and palladous sodium chloride. The preferred palladium salts are palladium chloride, palladium bromide, palladium iodide, palladium nitrate, palladium oxide and palladium sulfide. The most preferred palladium salt is palladium chloride.

While palladium or a salt thereof is required in the practice of the present invention, the complex may also further comprise an additional metal compound such as vanadate, molybdate, zinc or tin, or other cations such as potassium or sodium.

Oxidized and reduced forms of the complex are also contemplated. Whether the oxidized or reduced form is favored will depend upon the pH of the particular solution containing the complex.

### Long Chain Fatty Acid

The complex of the invention contains a long chain fatty acid. Any long chain fatty acid can be utilized in the complex, as long as the fatty acid is able to bond effectively to the palladium via its carboxyl end, and bond to zinc via its methyl end. Preferred fatty acids include, for example, linoleic acid and docosahexaenoic acid.

### Zinc

The complex of the invention may contain zinc bonded to the methyl end of a long chain fatty acid. The zinc can also bond to one or two amino acid residues. Zinc, according to the invention, can be in the form of elemental zinc, or derivatives of zinc including, for example, zinc carbonate, zinc gluconate, zinc chloride, zinc pyrithione, zinc sulfide, zinc methyl or zinc diethyl.

### Amino Acids

The complex of the invention may include one or two amino acid residues that are bonded to the zinc component of the complex. The amino acid residue can be any amino acid residue or derivative thereof. For purposes of this application, amino acid derivatives include naturally occurring derivatives and non-naturally occurring derivatives, i.e. synthesized amino acids.

A preferred amino acid is threonine. If two amino acids arc bonded to the zinc in the complex, they can be two of the same amino acid or two different amino acids.

### Pharmaceutical Compositions

The present invention also includes pharmaceutical compositions comprising the novel palladium-lipoic acid complexes as previously described supra. In general, the pharmaceutical compositions of the present invention may be administered by any enteral or parenteral route.

Parental administration includes intravenous, intramuscular, subcutaneous, intra-dermal, topical, intra-thccal and intra-arterial methods. Enteral administration includes any suitable form for oral consumption including, for example, tablets, pills, liquid gels, capsules, elixir, and troches.

A pharmaceutically acceptable carrier, according to the present invention, is any suitable carrier known to he skilled artisan and will depend upon the dosage form selected. Different routes of administration necessarily require different pharmaceutically acceptable carriers. An identification of such carders may be found in any standard pharmacy text, for example, Remington's Pharmaceutical Sciences, 17th edition, ed. Alfonso R. Gennaro, Mack Publishing Company, Easton, Pa. (1985).

More specifically, examples of pharmaceutically acceptable carriers include pharmaceutical diluents, excipients or carriers suitably selected for the intended route of administration which is consistent with conventional pharmaceutical practice. For instance, for oral administration in the form of tablets or capsules, the active drug components may be combined with any oral non-toxic pharmaceutically acceptable inert carrier such as starch, cellulose, magnesium stearate, dicalcium phosphate, calcium sulfate, mannitol, and the like. Moreover, when desired or necessary, suitable binders, lubricants, disintegrating agents and coloring agents can also be incorporated in the mixture. Suitable binders, for example, include starch, gelatin, natural and synthetic gums such as acacia, sodium alginate, carboxymethylcellulose, polyethylene glycol and waxes. Among the lubricants, there may be mentioned for use in these dosage forms, boric acid, sodium benzoate, sodium acetate, sodium chloride, etc. Disintcgrators include, without limitation, starch, methylcellulose, agar, bentonite, guar gum, etc. Flavoring agents and preservatives can also be included where appropriate. In the case of tablets, they can be further coated with the usual coating materials to make, for example, sugar-coated tablets, gelatin film-coated tablets, tablets coated with enteric coatings, tablets coated with films or double-layered and multi-layer tablets.

For parenteral administration, for example, the formulations must be sterile and pyrogen-free, and are prepared in accordance with accepted pharmaceutical procedures, for example as described in Remington's Pharmaceutical Sciences at pp. 1518-1522. The aqueous sterile injection solutions may further contain anti-oxidants, buffers, bacteriostats, isotonicity adjusters and like additions acceptable for parenteral formulations. Various unit dose and multidose containers, e.g., sealed ampules and vials, may be used, as is well-known in the art. The essential ingredients of the sterile parenteral formulation, e.g., the water and the selected palladium-lipoic acid complex, may be presented in a variety of ways, just so long as the solution ultimately administered to the patient contains the appropriate amounts of the essential ingredients. Thus, for example, the palladium-lipoic acid complex/water formulation may be presented in a unit dose or multidose container, ready for injection. As another example, a concentrated solution of palladium-lipoic acid complex/water may be presented in a separate container from a diluting liquid (water or palladium-lipoic acid complex/water) designed so that the contents can be combined to give a formulation containing appropriate amounts for injection. As another alternative, the palladium-lipoic acid complex may be provided in a freeze-dried condition in one container, while a separate container contains diluting liquid (water or palladium-lipoic acid complex/water, depending on the amount of palladium-lipoic acid complex in the other container), again designed so that the contents can be combined to give a formulation containing the appropriate amounts of the water and selected palladium-lipoic acid complex. In any event, the contents of each container will be sterile. Suitable carriers for parenteral administration include, for example, water, ethyl alcohol, propylene glycol, ethoxylated isostearyl alcohol, polyoxylated isostearyl alcohol, polyoxyethylene sorbitol and sorbitate esters. In these instances, adequate amounts of sodium chloride, glucose or glycerin can be added to make the preparations isotonic.

The dosage of the compositions of the present invention is selected, for example, according to the usage, purpose, conditions and symptoms. Furthermore, the dose administered will be selected, for example, according to the particular composition employed and the size and condition of the patient as well as the route of administration employed, but in any event will be a quantity sufficient to cause a reduction in tumor size.

An effective amount of the palladium lipoic acid complexes of the invention is, for example, an amount that results in inhibition of tumor growth and/or reduction in tumor size. For example, when the pharmaceutical composition of the present invention is parenterally administered to a patient, a dosage of between about 5 and about 30 ml daily of a 0.04 M solution of the pharmaceutical composition for at least about 5 days is employed. A contemplated dosage pattern in adult humans is about 40.0 ml of 0.04 M of the palladium lipoic acid complex administered daily for the first three days of treatment, followed by 20.0 ml daily for an additional 14 days of treatment. However, the precise route of administration, dosage and frequency of administration is individualized for each patient and can vary over a wide range depending on the particular disease state being treated, the condition of the patient and the like.

Higher dosages of the palladium-lipoic acid complexes, can be generally administered intravenously, while lower dosages may be given orally or by any injectable route.

A mammal that can benefit from treatment with the novel palladium complexes of the invention, as discussed supra, includes any mammal in need of reduction or elimination of a tumor or cancer. For example, suitable mammals include humans, domestic animals such as cats and dogs, and farm animals such as pigs, horses and cows.

In a method for inhibiting or reducing a tumor, the palladium complexes of the invention can be employed as a stand alone therapy, or in conjunction with other treatments such as, for example, radiation therapy and/or other chemotherapeutics.

### EXAMPLES

### Example 1

### Making the Complex

The palladium-lipoic acid complex of the present invention may be synthesized by first obtaining a palladium lipoic acid complex as disclosed in U.S. Pat. Nos. 5,776,973, 5,679,679 and 5,463,093, disclosures all of which are hereby incorporated by reference into the instant application.

A exemplary process for obtaining a complex according to the invention:

[1] The first stage is the solubilization of palladium dichloride in hydrochloric acid.

[2] The second stage is solubilizing lipoic acid in alkali.

[3] The third stage is reacting the solutions obtained from [1]and [2].

[4] A fourth stage is the dilution of [3] as a feedstock solution for synthesis of the final complex.

[1] Palladium dichloride powder (99.9% purity) is obtained from Alfa Aesar or DeGussa. Analytical grade HCl and NaOh are obtained from Baker. Water is deionized. All metal surfaces involved in the reaction are plastic coated.

A solution of 160.0 ml of 1.0 N HCl is placed in a 2 liter round glass reactor in a hemispheric heater. 14.20 g PdCl₂ is added to the HCl solution. The reactor vessel is stirred with a lightning type motorized stirrer having a plastic coated shaft and rotor so that no metal is exposed to the solution. Motorized stirring is continuous. When all the material is suspended the heat is turned on and the mixture is brought to a gentle boil. At boiling, stirring and heating arc continued for ten minutes. The boiling temperature is close to that of water, e.g.,100 degrees C.

After ten minutes of boiling a clear dark amber solution is produced. The material is allowed to cool overnight. The material is then filtered free of insolubles using a Buchner funnel with a fiberglass membrane. The solution is considered to be PdCl₄.

[2] In a separate similar reactor, 16.52 g of (DL) alpha lipoic acid (Sigma) is stirred into 285.0 ml of 2.0 N NaOH solution. 200.0 ml of water is added. The solution is stirred vigorously until it becomes a clear yellow. If any undissolved residue remains it is filtered to clarity.

[3] The PdCl₄ solution from [1] is added to the round flask of yellow lipoic acid solution from [2] and stirred thoroughly. The material is stirred continuously and brought to a gentle boil. Boiling temperature is close to that of water, e.g.,100 degrees C. Boiling is continued for fifteen minutes, producing a clear dark reddish brown solution. The material is allowed to cool overnight.

Sufficient water is stirred into the cooled solution to achieve a final volume of 597.0 ml. This is a 0.134 M solution of the complex of palladium-lipoic acid.

[4] The original PdLA stock solution method [3] is used to make sufficient quantity for the feedstock method.

The original PLA stock solution at 0.134 M will be diluted as follows to make 0.06 M. Since 0.06/0.134=0.447

447 ml. of 0.134 M PdLA are placed in a 2 Liter beaker over a magnetic stirrer. Sufficient purified water is added to achieve 1000 ml.

The solution is mixed well and bottled.

### Method for 1 Liter of Arginine-Linoleic Acid Feedstock Solution

All reactants are reagent grade.

In a 2. Liter beaker with a magnetic stirrer—dissolve 11.0 g L-arginine base (Sigma) in 800. ml purified H2O by stirring. add 16.8 g. linolcic acid (Alfa Aesar) with vigorous stirring for five minutes until a smooth gel is obtained.

Add 11.25. ml 6. N NaOH (Baker) and stir until clear.

Add sufficient water H2O to make 1000. ml. and stir until clear.

Refrigerate.

### Method for 1 Liter of Zinc Tri-Threonine Feedstock Solution

All reactants are reagent grade.

Zinc dichloride (Sigma) solution is prepared as follows—Since 50.0 mg/ml ZnCl2=0.365 M

50. g ZnCl2 are weighed and transferred to a 2 Liter beaker over a magnetic stirrer.

Purified water is added to achieve 1 Liter.

The solution is stirred vigorously until clear.

A portion of this solution will be used for the next step.

For a 0.06 M Feedstock solution, the 0.365 M ZnCl2 solution must be diluted—0.06 M/0.365 M=0.164

Measure 164.0 ml from the liter of 0.365 M ZnCl2 solution.

Weigh 28.6 g. of L-threonine.

Add threonine to 300 ml purified water in a 2 Liter beaker over a magnetic stirrer.

Stir to dissolve threonine.

Add the 164 ml. of ZnCl2 solution to the threonine solution and stir thoroughly.

Add water to approximately 900 ml. total volume and stir well.

Slowly add 21.0 ml of 6 N NaOH and stir well.

Add water to final volume of 1. Liter.

pH should be near 7.4

Refrigerate

### To Assemble the Three Major Components

A water-jacketed glass boiler arrangement is made to prevent foaming and over-boiling during the process.

Equal volumes of the three 0.06 M feedstock components are to be assembled.

However at the time of assembly, portions of each feedstock solution will be diluted with an equal volume of water to decrease the concentrations from 0.06 M. to 0.03 M.

These will be assembled in a water jacketed beaker over a magnetic stirrer-hot plate.

A 1 liter beaker is placed inside a 4 liter beaker and a perforated plastic spacer is placed on the bottom between the two beakers.

For this example, pour 200. ml of 0.03 M PdLA into the inner 1 liter beaker.

Next add 200. ml of 0.03 M arginine-linoleic acid solution to this beaker.

Place water in the outer beaker to reach the level of the two aliquots in the inner beaker.

Place a magnetic stir bar in the inner beaker and turn on the heater and stirrer.

An electronic thermometer probe is placed in contact with the inner beaker liquid surface.

Place a watch glass over the outer beaker.

Bring the outer water jacket to a gentle boil.

Allow the temperature of the inner liquid to rise to above 200 degrees Fahrenheit and maintain this temperature for six minutes.

Turn off the heat and allow to cool.

After five minutes, turn off the stirrer.

Allow system to cool to room temperature. This intermediate is called G10.

After thorough cooling add 200. ml. of 0.03 M Zinc tri-threonine to the G10 solution.

Turn on the stirrer.

Adjust the pH to 9.0 with 6.N NaOH.

Turn on the heater and bring the outer jacket to a boil.

Bring the inner liquid to above 200 degrees Fahrenheit.

Maintain temperature above 200 degrees Fahrenheit for six minutes.

Turn off the heat. After five minutes, turn off the stirrer.

Allow cooling to room temperature overnight.

Decant the liquid product.

Preserve the precipitate to calculate the yield of soluble product.

Take the pH. Slowly re-adjust pH to 9.0 with stirring using 1 N.HCl or 1 N.NaOH.

Add purified water to restore the volume lost in boiling to a final volume of 600. ml.

Microfilter—first through a 0.45μ pore, and then a sterile membrane system at 0.2μ pore.

Store in scaled sterile glass vaccine bottles with air excluded. This complex is referred to as G10Z.

Sufficient water should be added to the final solution to obtain a concentration of the complex of at least about 0.01 M, preferably between about 0.01 M and about 0.08 M, and most preferably in an amount sufficient to obtain a concentration of about 0.04 M in the resulting composition.

### Example 2

### Characterization of the Complex

Seven methods are used to characterize the complex obtained via the above-discussed protocol: (i) UV-Visible spectroscopy, (ii) FTIR spectroscopy, (iii) Cyclic Voltammetry, (iv) single frequency Impedance Spectroscopy (Mott-Schottky analysis), (v) Electron Spin Resonance Spectroscopy, (vi) HPLC, and (vii) Electrophoresis.

### UV-Visible Spectroscopy

A Shimadzu model UV160U double beam Spectrometer is used to acquire spectra of G10Z solution.

The stock solution of 0.01 M G10Z is diluted 1:500 for analysis and placed in a 1. cm. cuvet.

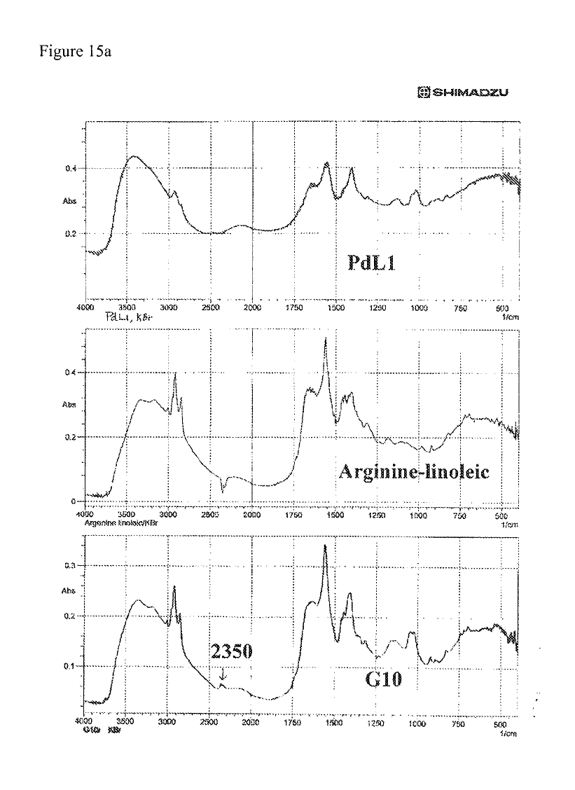

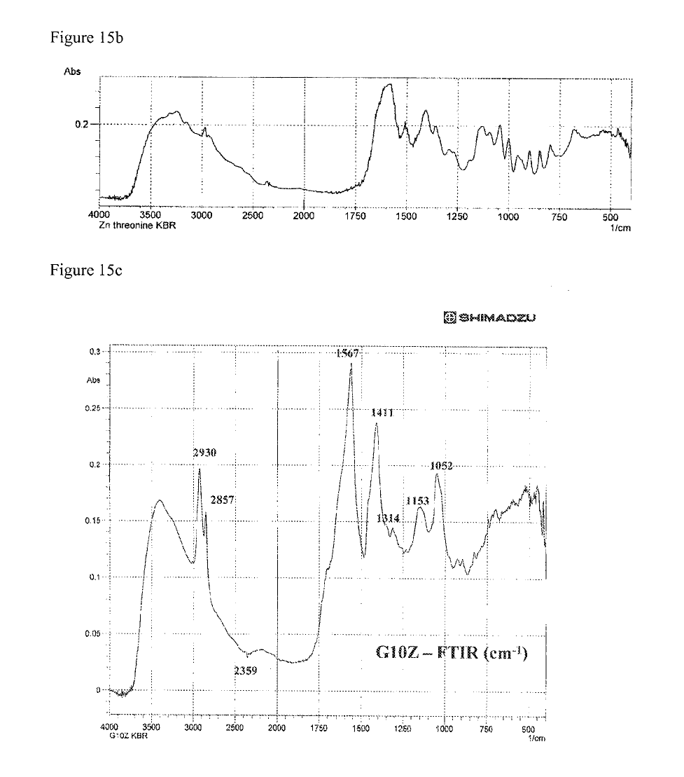

The spectra are shown with peaks at 237.5 and 284 nm.(FIG. 15). The molar absorbances for these respective wavelengths are 11,450 and 9,350.

### FTIR

### Methods

A Shimadzu model 8400S Fourier Transform Infrared Spectrophotometer is used to examine G10Z, and the three fractions or complexes from which it is derived. Samples are prepared by desiccation under vacuum and subsequently mulling 1 part sample by weight with 50 parts KBr. The three feedstock complex solutions are treated in the same way.

### Results

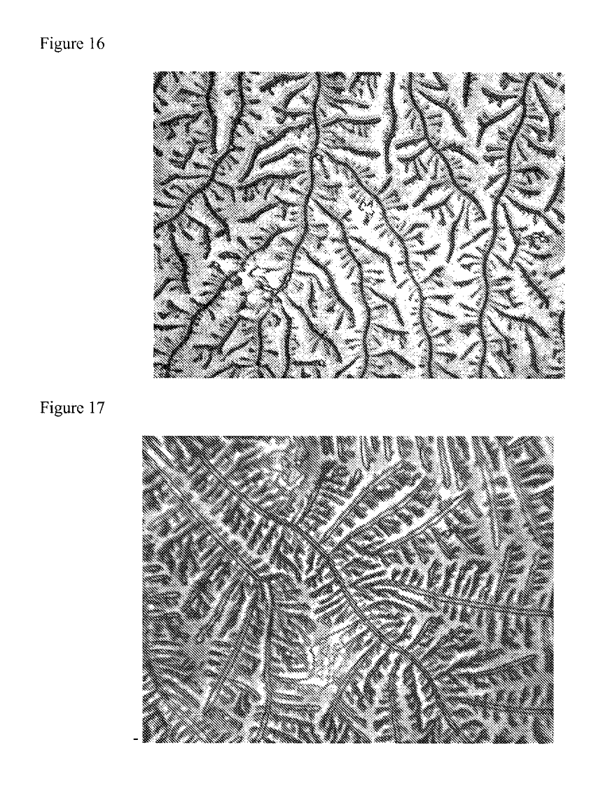

Diminution of a minimum in the arginine-linoleic acid complex at 2359 cm⁻¹ occurs after its reaction with the palladium lipoic acid component (FIG. 16a), to produce G10. Characteristic peaks occur in G10 at 750 and 2350 cm¹. G10 peaks at 750 and 2350 cm⁻¹ are suppressed in G10Z.

When stage G10 is reacted with the third feedstock solution, a stretched band arises at 1567 cm⁻¹. This G10Z band represents an amide stretch NH with secondary C−N (21), presumably from the amino acid threonine (FIG. 16b) associated with the zinc.

Representative peaks (cm⁻¹) for G10Z include : 1052 (CO stretch, also OH deformation (28)), 1153 (C—C stretch (29)), 1314 (CH2 wag(30)), 1411 (coordinated CO (31)), 1567 (amide stretch (21)), 2359, 2857 (CH stretch (32)), and 2930 (asymmetric CH3 stretch)(27).

The 2930 cm⁻¹ asymmetric CH3 stretch suggests the CH3 terminus of linoleic acid may be associated with the zinc in a self-assembling reaction (25).

The 1052 cm⁻¹ CO stretch and OH deformation band is a candidate for a linoleic acid carboxylic association with palladium with water librational modes (26). This is supported by the 1411 cm⁻¹ coordination CO band.

### Electrochemistry

### Voltammetry

An EG&G Parstat Model 2263 analytic potentiostat system is used with a gold working electrode, a platinum counter electrode and a AgCl—KCl reference electrode. Background electrolyte is 100. mL of 0.1 M. NaCl and the system is purged with nitrogen for eight minutes and a baseline established. Purging is performed for each addition.

The cyclic voltammetry scans are from −1.0 Volts to +1.50 Volts.

Calf thymus DNA is obtained from Sigma.

A G10Z signature is obtained using 1.0 mL. 0.01 M stock G10Z, and subsequently 1.0 mL. DNA is added from a solution containing 5.0 mg/mL DNA,

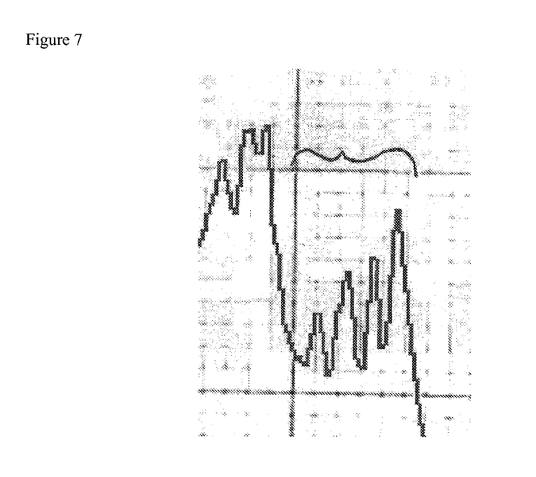

Scans are shown (FIGS. 7-a, b). It is evident that there is a peak shift representing an electron transfer of 60. mv. from G10Z to DNA. This charge transfer appears as irreversible.

### Mott-Schottky Impedance Analysis

The instrumentation and reagents and amounts here are as with the voltammetry.

The electroanalytic potentiostat system is used to scan the same voltage band at single perturbation frequencies from 50-200 mHz and observe the resulting inductances (FIG. 5).

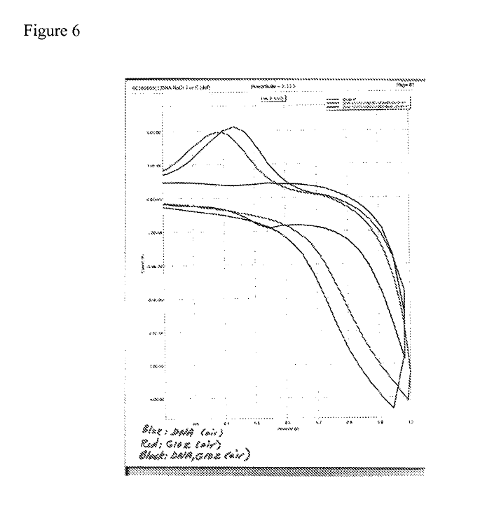

The inductance band is modified by the addition of DNA (FIG. 6).

These appearances of inductance oscillation arc unusual. The oscillations are consistent with the presence of repetitive subunit structure.

### Electron Spin Resonance Spectroscopy (ESR)

ESR is performed on the reaction of G10Z/vitamin B12, with DNA, using a continuous wave ESR X-band Spectrometer (Resonance Instruments Model 8400).

Equal volumes of 0.01 M. G10Z/with vitamin B12 (1.0 ug/ml. in the G10Z solution), DNA (Sigma et) solution (5.0 mg/ml), and 0.1 M NaCl, are mixed in a test tube. The mixture is loaded into NMR tubes and frozen overnight.

The test sample is transferred directly into the instrument from the freezer for scanning.

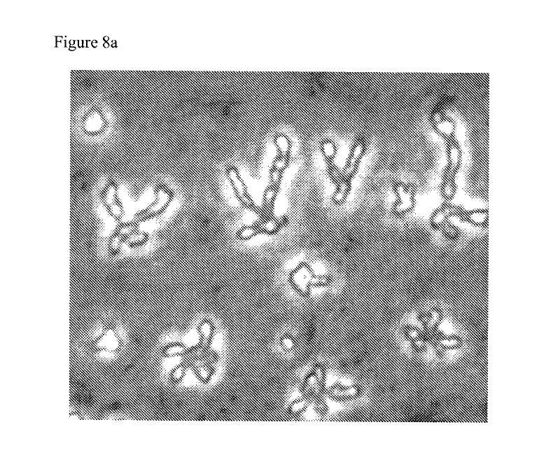

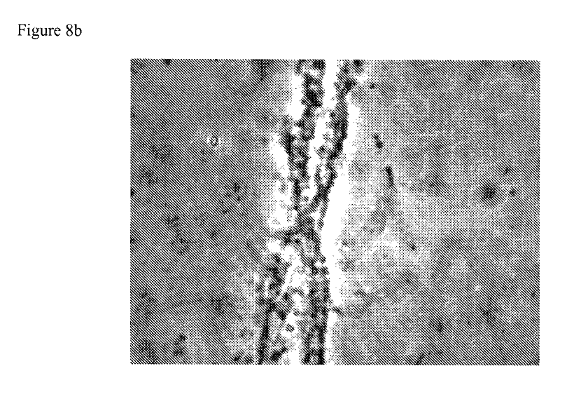

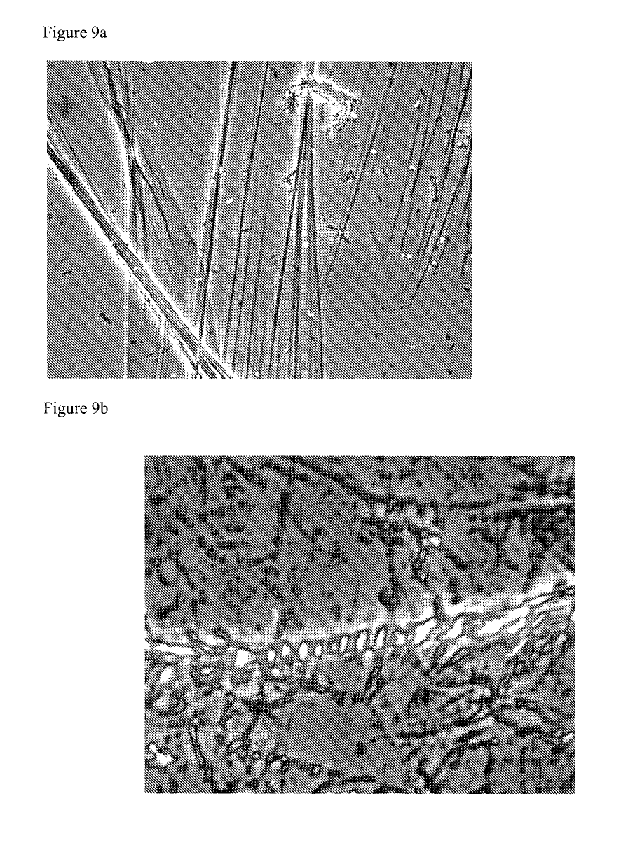

The results shown (FIG. 8) reveal quartets of symmetric peaks with a splitting constant of 6.5 Gauss (FIG. 8). This 6.5 Gauss value matches a constant observed by Gordy and Alexander in their ESR studies of irradiated single crystals of guanine (12).

An excited state of guanine in DNA would allow stacking within DNA and fits the theoretical models of Barton (20) and Hennig (13). Hennig's theory argues that base stacking enabled by charge, causes a configurational supercoil shift in DNA, and the charge is then transferred to the hydrogen bonding sites of the bases. Charge is then transferred into the long axis of the helix. The configurational shift during DNA charge transfer is also supported by the work of Schuster (22). Therefore the ESR reaction supports a dynamic configurational change in DNA.

### HPLC

High Performance Liquid Chromatography is performed with a Beckman-Coulter System

Gold. The method is as follows:

0.1 M ammonium sulfate isocratic run for ten minutes.

The G10Z retention time is from 0.5 to 1.5 minutes (FIG. 24). This band demonstrates

G10Z as a large peak with a small close peak comprising excess threonine.

### Electrophoresis

A Novex electrophoresis sytsem is used with Nupage gel plates. A tris-glycine sodium dodecyl sulfate running buffer is used.

The dense brown color of G10Z makes it easy to track and identify as the only palladium fraction.

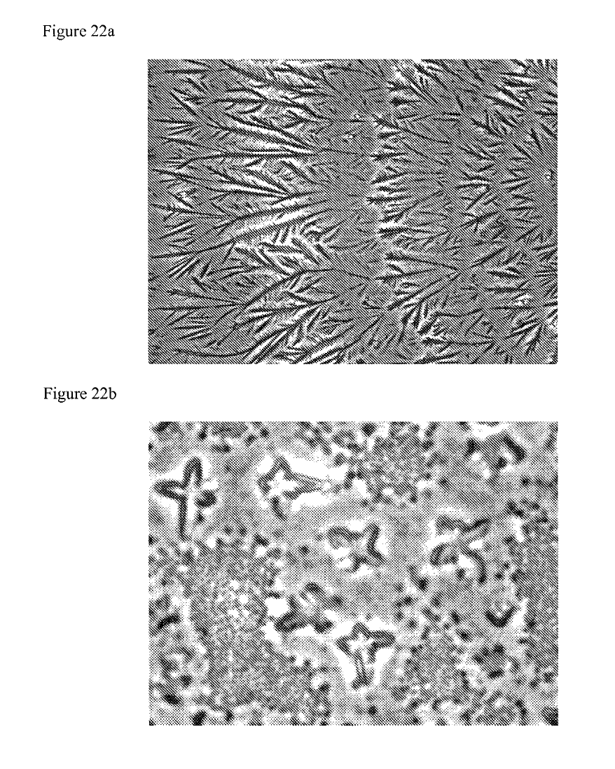

Molecular weight markers are run in tandem with G10Z. The results are shown (FIG. 22). The molecular weight of G10Z is close to but less than the nearest marker which is bradykinin which has a molecular weight of 1060. Therefore G10Z weighs less than 1060.

A calculation from the weight of G10Z constituents is as follows: palladium+lipoic acid+linoleic acid+zinc+2 threonines=897. molecular weight.

### Example 3

### Mechanisms of Action—Rationale of Structure

### Biochemical Effects—Liquid Crystal Nature

Voltammetry shows the non-reversible transfer of charge from G10Z to DNA (FIG. 7-a,b). The amount of the transfer is 60.0 millivolts.

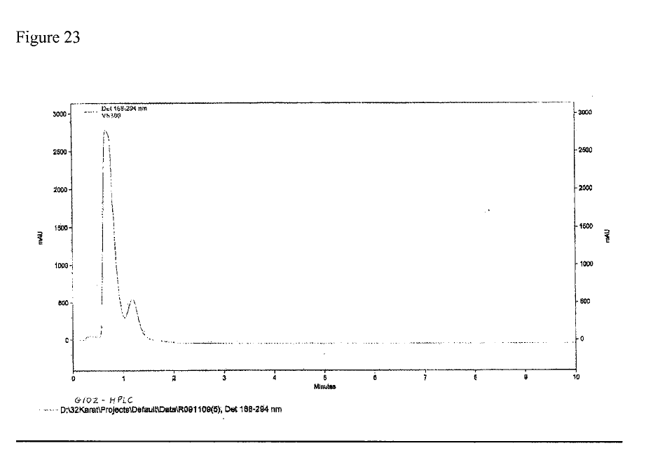

In electron spin resonance spectroscopy a spin quartet is manifested by G10Z molecular hyperfine interaction with DNA (FIG. 8). This behavior is believed to derive from the spin properties of palladium. This view derives from images showing periodic crystal packing of palladium (FIG. 23a) in which PdCl4 is neutralized by excess NaOH. It is believed the packing geometry is derived from palladium d-orbital spin effects which extend through the bound water.

G10Z in NaCl shows liquid crystal formation (FIG. 23b). The DNA liquid crystal fern structure is demonstrated to occur in 0.1 M NaCl (FIG. 16). Such structures associated with spin signals introduce the phenomena of liquid crystal lattices and associated Bloch waves and are reported for PLA and DNA (16). These mesoscopic states provide a lattice model for interactions.

The original Pd-lipoic acid complex provides a structure in which the palladium is safely sequestered, and which is also active in the lipoic acid binding site in mitochondrial complex I, thereby coupling to enzymatic charge transfer. The bound addition of linoleic acid to Pd-lipoic acid moves its standard potential into the electro-positive (+) range, and increases the anti-tumor activity in test systems. However the complex at this G10 stage is so electrically stimulating as to cause the incidence of seizures in mice.

### Example 4

### Modification By Zinc Complex

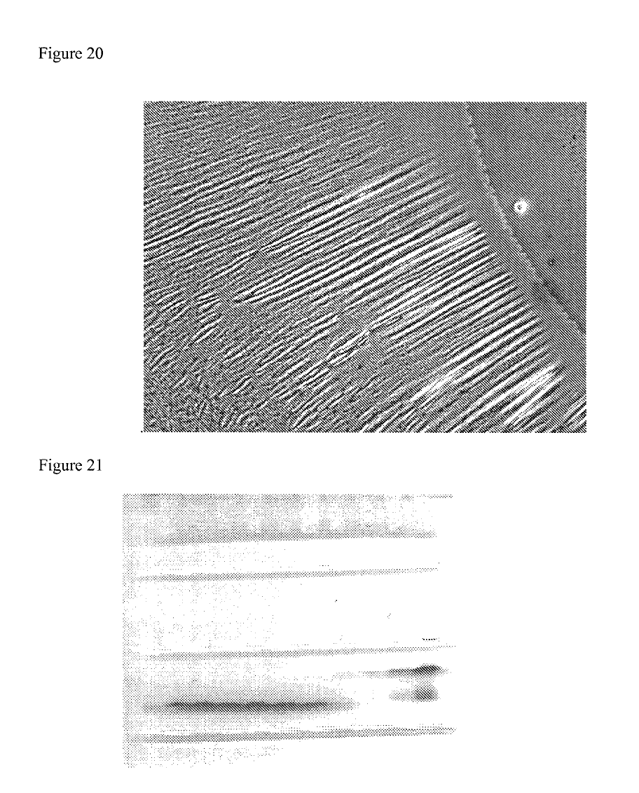

To eliminate seizure activity, the G10 stage of the synthetic complex was further investigated and combined with various substances, until there were no seizures. This was ultimately achieved by complexing with zinc-amino acid complexes. Zn-bis(threonine) which has a highly polarized liquid crystal structure (FIG. 20), modulates and inhibits the seizure potentials in mice.

The zinc coordination number of three amino acids is noted in the literature. Some excess of threonine is included in the synthesis to increase collision frequency and insure coordination of the metal.

### Example 5

### Primitive Living Systems and Also Biological Polymers are Also Investigated for Further Influences of G10Z

The living systems studied are Bakers' yeast (Saccharomyces cerevisae), and the mold Dictyostelium discoideum. The biological polymers are calf thymus DNA (ct), poly GC (polydeoxy guanidylic/polydeoxy cytidylic), and DNA (ct) with histone ITS.

### Mold Species

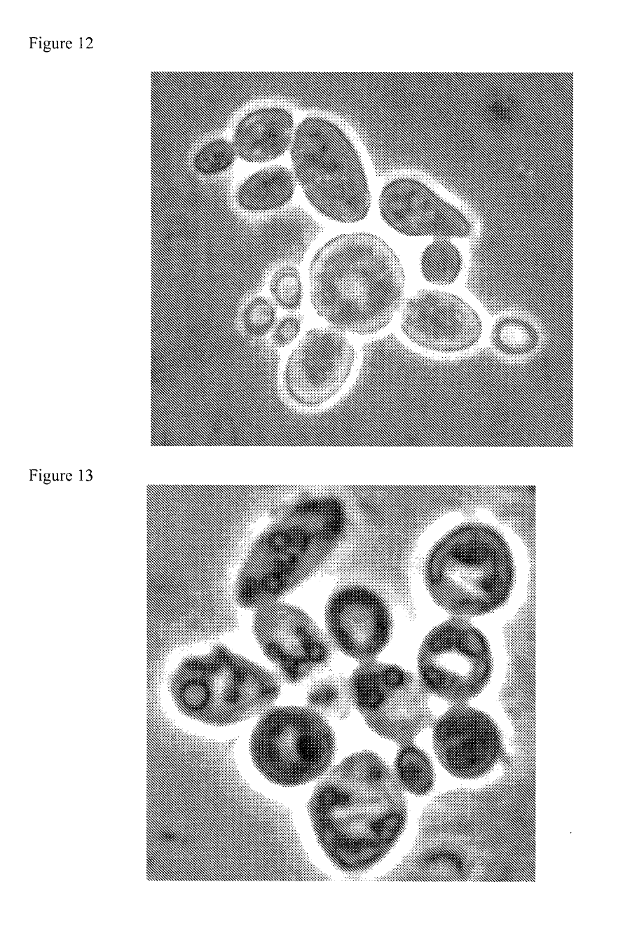

Dictyostelium discoideum is obtained from Carolina Biological and the agar plates are immediately viewed under stereo microscopy. The growth pattern appears as a vine-like structure showing spores (sori) as swollen fruit-like structures (FIG. 11). Some of the plates are inoculated with a thin layer composed of 2.0 ml. of 0.005 M G10Z solution. The plates are kept covered and at room temperature. After three days the inoculated plates are re-examined and show a new pattern of dense growth. This new growth is in the form of numerous spores growing from a common trunk so as to resemble fruit trees in an orchard (FIG. 12). This vigorous development pattern is a distinct departure from the unchanged control plates.

### Yeast Cells

Bakers' yeast is obtained from Carolina Biological and cultured in 5% malt extract with 5% sucrose. These are allowed to grow out three days in plastic flasks. 10 l samples are placed on glass slides under cover slips and inoculated with G10Z to 10⁻⁶M. final concentration.

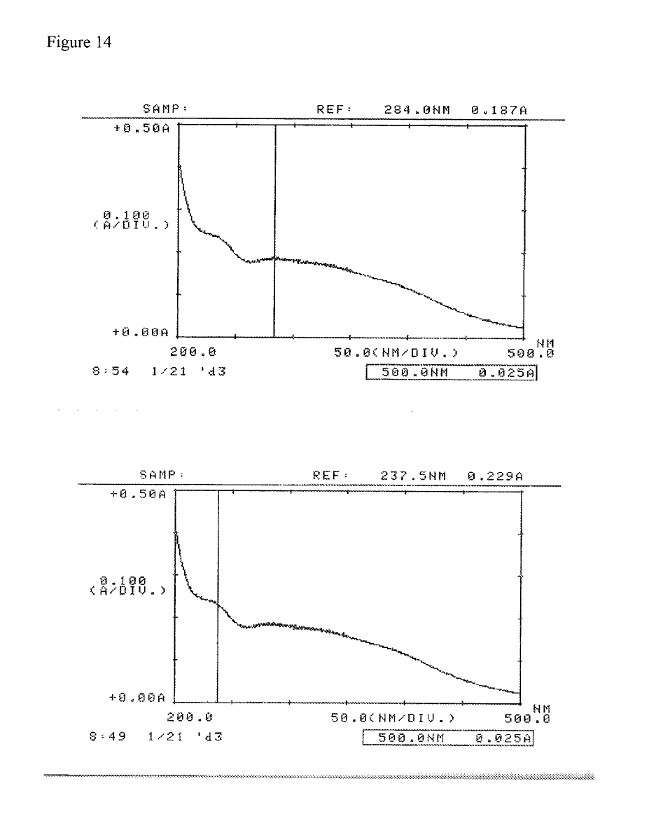

While under observation by phase microscopy, the G10Z produced heterochromatin (dense chromatin). (FIGS. 13 and 14). Heterochromatin is a model for gene repression (14).

### Example 6

### Influences of G10Z on Gene Polymer Liquid Crystal Configurations

### 1. DNA

Microscope slides are prepared to receive 10 μL. of the following mixture: 10 parts.1 M NaCl, 10 parts 5.0 mg/mL.DNA (ct) solution, and 1 part 10⁻²M G10Z solution.

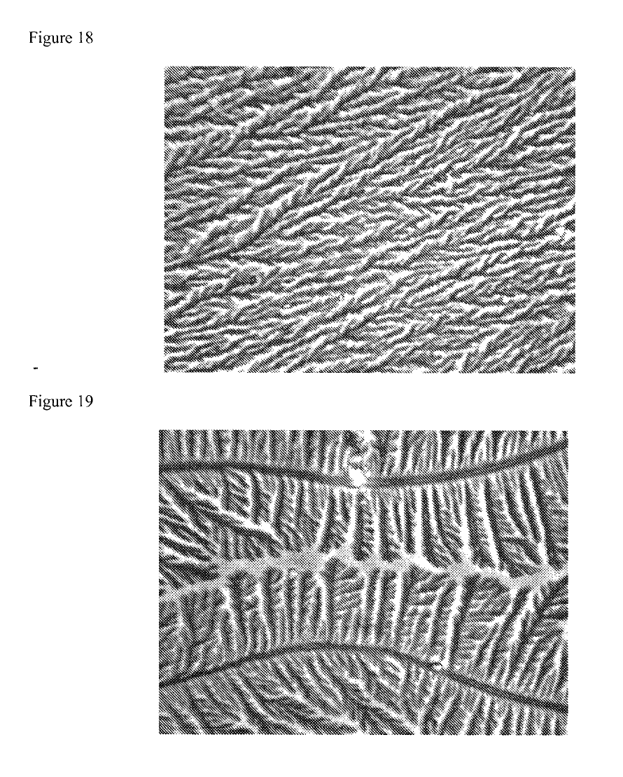

A 10 μL aliquot of the mixture is spread evenly over the entire slide with a loupe and allowed to dry. The dried slide is viewed under phase microscopy (FIG. 18) and compared with a control slide which is similarly prepared but receives no G10Z (FIG. 17). Initially one can see a small intrinsic waveform in the DNA liquid crystal. The G10Z increases this wavelength in a form of Doppler influence. We interpret the mechanism as one that converts a self-inductance to a mutual inductance or resonance.

### 2. Poly GC

Microscope slides are prepared to receive 10 μL of the following: The prepared mixture contains 10 parts 0.1 M NaCl, 10 parts 5.0 mg/mL poly GC (poly deoxyguanidylic-polydeoycytidylic acid), and 1 part 10⁻²M G10Z solution. A 10 μL aliquot of the mixture is spread evenly over the entire slide with a loupe and allowed to dry. The dried slide is viewed under phase microscopy (FIG. 20) and compared with a control slide which is similarly prepared but receives no G10Z (FIG. 19). The control slide shows absence of waveforms. The G10Z slide shows large waveform structures induced in poly GC.

### 3. DNA With Histone

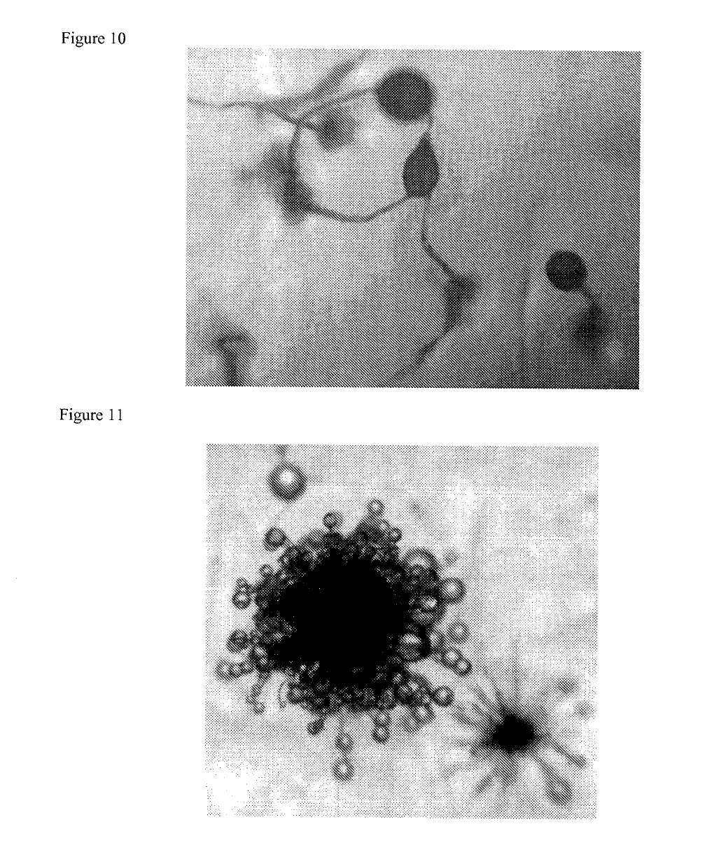

Microscope slides are prepared to receive 10 μL of the following: The prepared mixture contains. 10 parts 0.1 M NaCl, 10 parts 5.0 mg/ml DNA solution, 10 parts 5.0 mg/mL histone IIS solution, and 1 part 10⁻² M G10Z solution. A 10 μL aliquot of the mixture is spread evenly over the entire slide with a loupe and allowed to dry. The dried slide is viewed under phase microscopy and compared with a control slide which is similarly prepared but receives no G10Z. The control slide shows the formation of straight filaments (FIG. 10a). Only the G10Z treated slide (FIG. 10b) shows the induction of twisted cable structures—analogous to chromatin.

### Example 7

### Utility in Medicine—In Vitro and In Vivo Screening

An in vitro method was first utilized in the screening of G10Z as a candidate chemotherapy agent.

### Methodology of the In Vitro Cancer Screens

For a typical experiment, cells arc inoculated into 96 well microtiter plates in 100 μL at plating densities ranging from 5,000 to 40,000 cells/well depending on the doubling time of individual cell lines. After cell inoculation, the microtiter plates are incubated at 37° C., 5% CO2, 95% air and 100% relative humidity for 24 h prior to addition of experimental drugs. Aliquots of 100 μl of the different drug dilutions are added to the appropriate microtiter wells already containing 100 μl of medium, resulting in the required final drug concentrations.

Following drug addition, the plates are incubated for an additional 48 h at 37° C., 5% CO2, 95% air, and 100% relative humidity. For adherent cells, the assay is terminated by the addition of cold TCA. Cells are fixed in situ by the gentle addition of 50 μl of cold 50% (w/v) TCA (final concentration, 10% TCA) and incubated for 60 minutes at 4° C. The supernatant is discarded, and the plates are washed five times with tap water and air dried. Sulforhodamine B (SRB) solution (100 μl) at 0.4% (w/v) in 1% acetic acid is added to each well, and plates are incubated for 10 minutes at room temperature. After staining, unbound dye is removed by washing five times.

After staining, unbound dye is removed by washing five times with 1% acetic acid and the plates are air dried. Bound stain is subsequently solubilized with 10 mM trizma base, and the absorbance is read on an automated plate reader at a wavelength of 515 nm. For suspension cells, the methodology is the same except that the assay is terminated by fixing settled cells at the bottom of the wells by gently adding 50 μl of 80% TCA (final concentration, 16% TCA).

Using the seven absorbance measurements [time zero, (Tz), control growth, (C), and test growth in the presence of drug at the five concentration levels (Ti)], the percentage growth is calculated at each of the drug concentrations levels. Percentage growth inhibition is calculated as:

[(Ti−Tz)/(C−Tz)]×100 for concentrations for which Ti>/=Tz

[(Ti−Tz)/Tz]×100 for concentrations for which Ti<Tz.

Three dose response parameters arc calculated for each experimental agent. Growth inhibition of 50% (GI50) is calculated from [(Ti−Tz)/(C−Tz)]×100=50, which is the drug concentration resulting in a 50% reduction in the net protein increase (as measured by SRB staining) in control cells during the drug incubation. The drug concentration resulting in total growth inhibition (TGI) is calculated from Ti=Tz.

The LC50 (concentration of drug resulting in a 50% reduction in the measured protein at the end of the drug treatment as compared to that at the beginning) indicating a net loss of cells following treatment is calculated from [(Ti−Tz)/Tz]×100=−50. Values are calculated for each of these three parameters if the level of activity is reached; however, if the effect is not reached or is exceeded, the value for that parameter is expressed as greater or less than the maximum or minimum concentration tested.

### Results—In Vitro

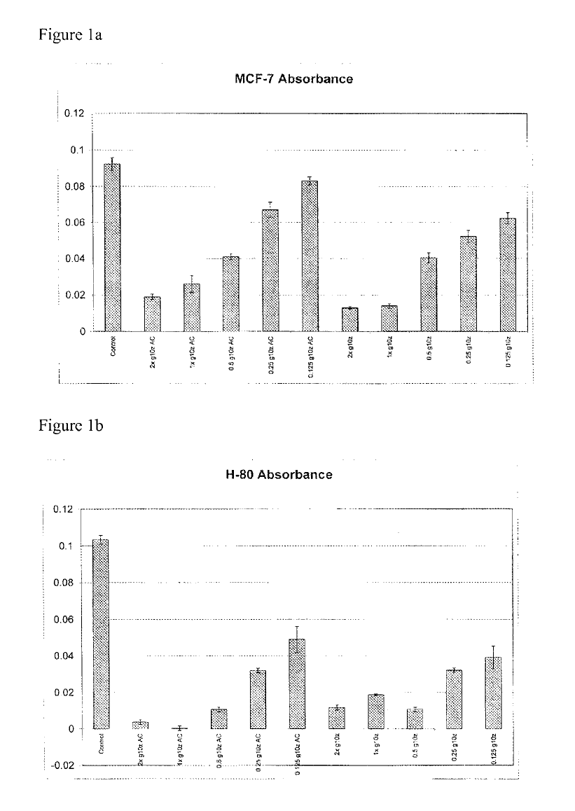

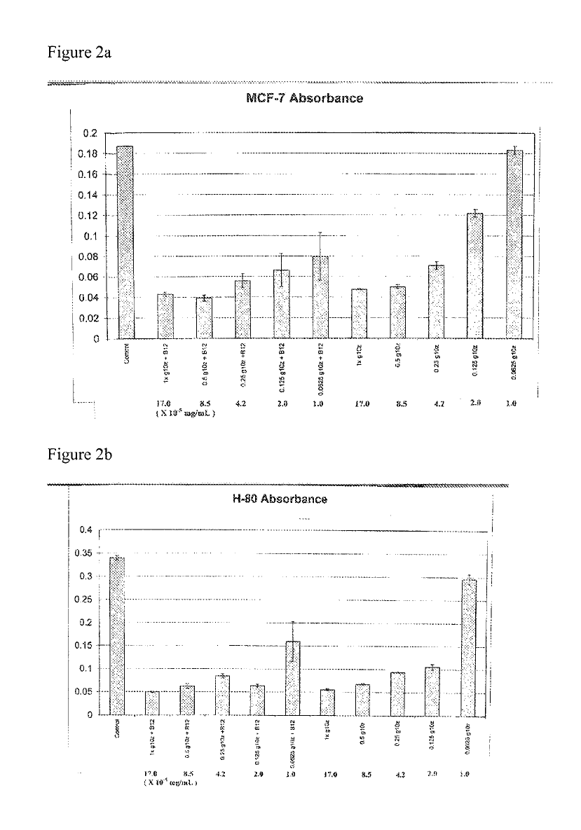

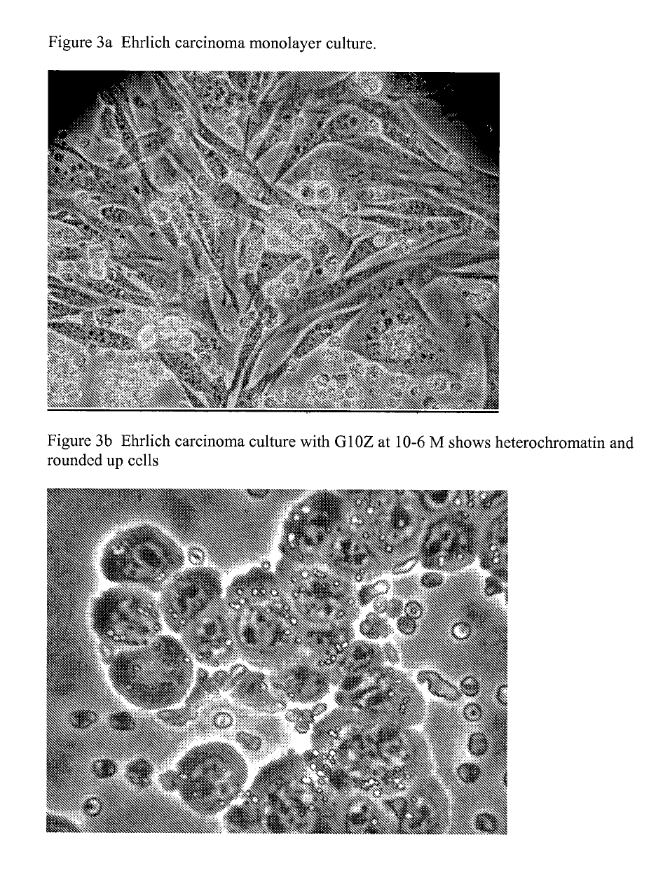

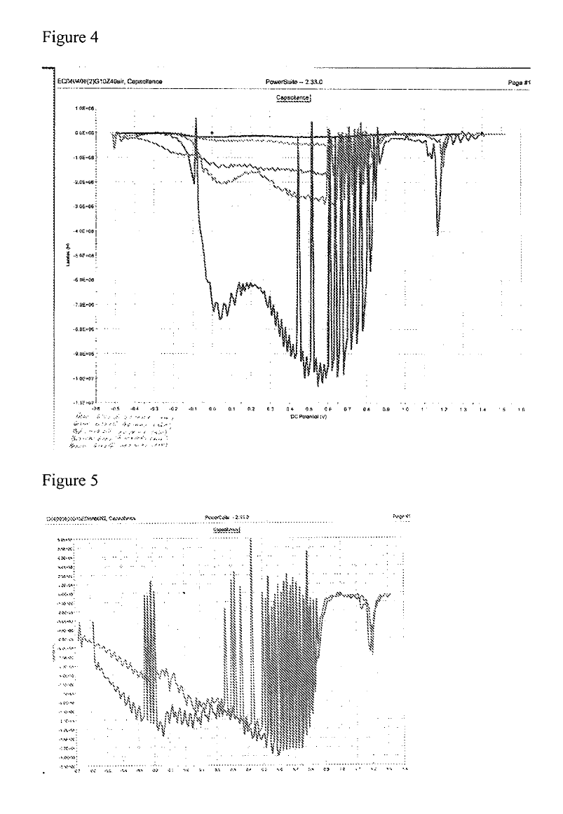

The tabulations of the in vitro effects of G10Z and G10AC show the cytotoxic effects of these agents on breast cancer and brain cancer cells (FIGS. 1a, b). The cytotoxicity shows as a function of concentration. The five test concentrations are graded as 3.0, 6.0, 12.0, 24.0, 48.0 μg/mL.

In another study (FIG. 2a, b), the G10Z concentration is graded as 1.0, 2.0, 4.3, 8.5, and 17.0×10⁻⁵ mg/ml. The B12 concentration is graded in 5 stages from 1×10⁻⁴ μg/ml to 17×10 ⁻⁴ μg/ml. At the lowest dose of G10Z the B12 shows a cooperative cyto-toxic effect.

### Method—In Vivo Methodology of Animal Cancer Screens

G10Z was studied with respect to Ehrlich ascites carcinoma obtained from the National Institutes of Health.

Ehrlich ascites carcinoma was established in Balb C mice. The ascitic serum was allowed to grow out to an amount that was easily withdrawn by syringe. The cells were examined microscopically to confirm viability and concentration. The serum was diluted 10× in Dulbecco's culture medium and 10⁶ cells were injected into the peritoneum of 12 test mice.

After 24 hours, a group of 4 of these mice was injected i.p. with 0.2 ml. 0.02 M G10Z, on a daily basis except for weekend lapses. A 19 day study was performed. Injections were performed on 14 of the days for a total of 14 injections, allowing weekend lapses on the 5^{th} and 6^{th} and 12^{th} and 13^{th} days. The remaining 8 mice served as untreated controls.

The results of the experiment are as follows:
Control: number of mice=8
Average days survived=13.75
Treated: number of mice=4
Average days survived=15.5

Summary—Preliminary data is consistent with a 12% increase in survival time in an ascites stage 4 cancer model.

1. A complex of Formula I
(Me)ₐ(Lipoic acid)_{b}(fatty acid)_{c}(zinc)_{d}(amino acid)ₑ (Formula I)
wherein
the Me component is palladium or a palladium salt thereof;
the lipoic acid component comprises lipoic acid or a derivative thereof, wherein the lipoic acid component comprises at least two sulfur atoms and at least one carboxyl group;
the fatty acid component comprises a hydrocarbon chain of from 2 to twenty carbon atoms wherein at one end of the hydrocarbon chain is a methyl group and at the other end is a carboxyl group;
the zinc component, when present, is bonded to a methyl group at one end of the fatty acid,
and the amino acid component, when present, is bonded to the zinc component,
the palladium is bonded to the lipoic acid component via both sulfur atoms and by the carboxyl group oxygens of the lipoic acid component;
the fatty acid is bonded to the palladium via the carboxyl group of the fatty acid;
wherein a, b, and c are each 1
d is 0 or 1
e is 0, 1 or 2
and standard potential of the complex is electropositive.

2. The complex of claim 1, wherein the palladium salt is selected from the group consisting of palladium chloride, palladium bromide, palladium iodide, palladium nitrate, palladium oxide and palladium sulfide.

3. (canceled)

4. The complex of claim 1, wherein the lipoic acid derivative comprises a side group selected from a carboxyl, a sulfur, an amine or a combination thereof.

5. The complex of claim 4, wherein the lipoic acid derivative is lipoamide:

6. The complex of claim 1, wherein the fatty acid is linoleic acid.

7. The complex of claim 1, wherein the fatty acid is docosahexaenoic acid.

8-10. (canceled)

11. The complex of claim 1, wherein when e is 2, and the complex comprises two amino acid residues, the two amino acids are the same amino acid or two different amino acids.

12. The complex of 11, wherein the two amino acids are threonine.

13-15. (canceled)

16. A pharmaceutical composition comprising a therapeutic amount of a complex according to claim 1, and a pharmaceutically acceptable carrier.

17-35. (canceled)

36. The complex of claim 1, wherein the zinc component is a form of zinc selected from elemental zinc, zinc carbonate, zinc gluconate, zinc chloride, zinc pyrithione, zinc sulfide, zinc methyl or zinc diethyl.

37. The complex of claim 1, further comprising at least one ligand selected from an inorganic anionic ligand or a cationic ligand.

38. The complex of claim 1, wherein the inorganic anionic ligand is acetate, acetylacetonate, amine, ammonium chloride, ammonium nitrate, bromike chloride, fluoride, iodie, nitrate, nitrite, oxalate, oxide, pyridine, sulfate or sulfide.

39. The complex of claim 1, wherein the lipic acid derivative further comprises a cation selected from sodium, potassium, magnesium, caldium, ammonia, vanadate, moybdate, zinc, and tin.

40. The complex of claim 1, wherein the complex is thermodynamically stable.

41. The complex of claim 1, wherein a Fourier Tranform infrared spectrum of the complex comprises a peak at 1052 cm−1; 1153 cm−1, 1314 (cm−1), 1411 (cm−1), 1567 (cm−1), 2359 (cm−1), 2857 (cm−1), and 2930 (cm−1).

42. The complex of claim 1, wherein a, b, and c are 1 and d and e are 0.

43. The complex of claim 1, wherein the complex is effective to transfer electrons from the complex to DNA or RNA.

44. The complex of claim 43, wherein transfer of electrons from the complex to DNA leads to condensation of chromatin.

45. The complex of claim 43, wherein the transfer is non-reversible.

46. The pharmaceutical composition of claim 16, wherein the complex is a solution or a dispersion.

47. The pharmaceutical composition of claim 45, wherein the complex in solution is present in an amount sufficient to obtain a concentration of about 0.04 M.

48. The pharmaceutical composition of claim 16, wherein the composition is administered enterally or parenterally.

49. The pharmaceutical composition of claim 16, wherein the therapeutic amount of the complex in the composition is cytotoxic to breast cancer cells.

50. The pharmaceutical composition of claim 16, wherein the therapeutic amount of the complex in the composition is cytotoxic to brain cancer cells.

51. The pharmaceutical composition of claim 16, wherein the therapeutic amount of the composition is cytotoxic to Ehrich ascites carcinoma cells.

52. The pharmaceutical composition of claim 16, wherein the therapeutic amount of the complex when d is 1 is without seizure activity, compared to the therapeutic amount of the complex when d is 0.
